(19) United States
(12) Patent Application Publication
(10) Pub. No.: US 2019/0001164 A1
(43) Pub. Date: Jan. 3, 2019

(54) METHOD FOR ADMINISTERING IMPROVED HYGIENE SERVICES AND BOTANICAL AND BIOMIMETIC TREATMENT OF DENTAL AND PERIODONTAL CONDITIONS

(71) Applicant: Sandra Senzon, New York, NY (US)

(72) Inventor: Sandra Senzon, New York, NY (US)

(21) Appl. No.: 15/991,806

(22) Filed: May 29, 2018

Related U.S. Application Data
(60) Provisional application No. 62/511,548, filed on May 26, 2017.

(51) Int. Cl. A61Q 11/00; A61K 8/92; A61K 8/04

(52) U.S. Cl. CPC A61Q 11/00; A61C 19/002; A61K 8/046; A61K 8/922

(57) ABSTRACT

A method for administering improved hygiene services and a botanical and biomimetic treatment of dental and periodontal conditions include applying an aerosol spray botanical composition to a patient's oral cavity, pre-treating instruments with clove oil, brushing with a herbal oil-infused brush to massage the patient's teeth and gums, which massaging tightens the gum tissue. Another method comprises rinsing a patient's oral cavity with one of a solution of sea salt or an oil pulling mouthwash, brushing the teeth and gums with a brush to which at least one herbal oil has been applied, flossing the teeth and gums, applying an aerosol spray to a quadrant of the patient's oral cavity, said oral spray preferably comprising a botanical composition such as a herbal oil or oils, and massaging the gums with the oil or oils.

### CROSS-REFERENCE TO RELATED APPLICATIONS

The present application claims priority under 35 U.S.C. § 119 on the U.S. Provisional Patent Application Ser. No. 62/511,548, filed on May 26, 2017, the disclosure of which is incorporated by reference.

### FIELD OF THE DISCLOSURE

The present disclosure relates to a biomimetic method of for administering oral care, and, more particularly, to method for administering improved hygiene services and a botanical treatment of periodontal conditions.

### BACKGROUND

Proper dental hygiene mandates that an individual regularly visit a dental office for routine cleaning and otherwise maintain proper care at home as often as three times a day. Caring for one's teeth generally involves brushing them with toothpaste, which may eliminate a substantial amount of bacteria present in the mouth and thus help prevent or reduce the risk of certain oral health issues such as tooth cavities and the gum disease gingivitis. However, even an individual who routinely cares for his or her teeth and gums may be subjected to other oral issues notwithstanding such care, which other issues may, for example, include tooth decay and gum disease.

Tooth decay and gum disease are conditions that are both uncomfortable and potentially unhealthy. Tooth decay often leads to cavities forming in teeth, which requires fillings (or worse, extractions) to address the decay conditions. To treat these conditions sufficiently, an individual must visit a dental professional. However, the visit to a dentist's office is often not a pleasant experience.

That is, a visit to the dentist invokes a sense of fear and anxiety among many Americans. This fear may come from a fear of pain, feelings of loss of control, embarrassment, or negative past experiences. An estimated 9-15% of Americans avoid seeing the dentist because of this fear, meaning that 30-40 million people do not visit the dentist merely because of their anxiety of going to the dentist. Those that forego their annual dental visit are then more likely to suffer from poor oral health, and thus poorer health in general.

There are a few mechanisms used by dental offices to improve comfort for visitors that may have such fear and anxiety. For instance, some dental offices try to make their seating more comfortable by buying seats with pads and movable armrests, so that patients can feel comfortable both while waiting and during their examination. Other offices provide magazines, colorful wall art, televisions, and even fish tanks to create an uplifting atmosphere that both adults and children may enjoy to take their minds off of their examinations.

Furthermore, existing treatment methods require administration of chemical-based preparations, which preparations at best involve non-natural ingredients and otherwise involve administration of potentially harmful chemical compounds to a patient, which contributes to patient anxiety, and potentially, adverse side-effects to the patient, as well. Dental instruments known in the art (such as scrapers and picks used to remove tartar and plaque) are abrasive and often result in inflaming the gums as part of the removal process. Causing inflammation as part of a hygiene program is ultimately contrary to the patient's interest and comfort. Further, traditional methods of prosothodontics and tooth repair (such as crowns, for example) require breaking up tooth structure further before the repair can be performed, i.e., to create a clean seat for the crown to be installed. Nor do these traditional methods do not take advantage of the body's own healing techniques.

As such, these existing solutions do not significantly reduce patient anxiety, let alone provide for an effective manner of treatment for dental conditions. Accordingly, there exists a need for a method for administering improved hygiene services and botanical treatment of periodontal conditions that minimizes patient anxiety and that avoids the use of non-natural ingredients that may otherwise cause unwanted side-effects in patients. Further, there exists a need for treatment that minimizes further destruction of tooth structure in connection with tooth repair and that mimics and/or utilizes the body's own healing techniques.

### SUMMARY OF THE DISCLOSURE

In view of the foregoing disadvantages inherent in the prior art, the general purpose of the present disclosure is to provide a method for administering improved hygiene services and a botanical and biomimetic treatment of dental and periodontal conditions to include all advantages of the prior art, and to overcome the drawbacks inherent in the prior art. It is an object of the present disclosure is to provide comfortable dental treatment to a patient that avoids the anxiety otherwise associated with traditional dental offices and to treat the patient with natural ingredients to cosmetically improve a user's oral tissue, and further to provide a composition for treatment that excludes chemicals known to cause dentinal sensitivity and pose other health risks. It is another object of the present methods to utilize and/or mimic the body's own healing techniques.

In an embodiment, a method for administering improved hygiene services and a botanical treatment of periodontal conditions comprises (1) inquiring of a patient as to whether the patient has any allergy to botanical products, (2) applying an aerosol spray to a quadrant of the patient's oral cavity, said oral spray preferably comprising a botanical composition such as a herbal oil, baking soda, sea salt, or aloe vera gel. The application of the spray to the patient is done prior to using dental instrumentation on the patient's teeth (to loosen plaque and tartar from the patient's teeth) or gums (to open the gum tissue). The spray loosens tartar that is on the teeth. The method then includes cleaning the patient's teeth and gums with traditional dental instrumentation (such as picks and scrapers). After instrumentation, the gum tissue may be left raw or irritated from the instrumentation. Accordingly and thereafter, a brush to which at least one herbal oil has been applied is used to massage the patient's teeth and gums, which massaging tightens the gum tissue, which restores and strengthens the gum tissue. The comfortability of the brush reduces patient anxiety and discomfort. The method is repeated for the remaining quadrants of the patient's mouth to complete the treatment.

The treatment of both oil and water mimics the human body's natural healing process, which amounts to a less invasive and less impactful treatment of the patient. The use of botanical oils mimics the body's own defense mechanisms (such as the body's production of antioxidants) by retaining and/or enhance moisture in the oral cavity and by acting as a shield against plaque and by maintaining or approaching the body's preferred pH balance in the mouth, which pH balance also rejects plaque retention. This benefit is particularly relevant as a response to a person's acid generating reaction to certain foods, as the presence of acid has the effect of retaining plaque on teeth. The shielding of the teeth by the botanical oils, as well as the oils' tendency to restore pH balance, reduce or eliminate plaque retention.

The botanical oils are surfactant-free, and are antioxidants and antimicrobials, while also not allowing the gum tissue to dry out or create sloughing of such tissue. As such, the method enhances the body's own processes of healing of gum tissue as well as the surrounding bone. The use of the botanical oils and the method disclosed herein therefore aid in mimicking the body's own defense mechanisms as well as the method by which the body produces such defensive compositions.

In another embodiment, a method for administering improved hygiene services and a botanical and biomimetic treatment of dental and periodontal conditions may be practiced by a patient at home (as opposed to being administered by a dental professional at a dental office). In such an embodiment, the method comprises (1) rinsing the oral cavity with one of a solution of sea salt or an oil pulling mouthwash, (2) brushing the teeth and gums with a brush to which at least one herbal oil has been applied, (3) flossing the teeth and gums, and/or cleaning the teeth with teeth sticks, (4) applying an aerosol spray to a quadrant of the patient's oral cavity, said oral spray preferably comprising a botanical composition such as a herbal oil or oils, and (5) massaging the gums with the oil or oils. In an embodiment, if gums are inflamed, the patient may optionally apply the oil to a piece of gauze and then wrap the inflamed area with the oil-infused gauze.

The methods disclosed herein provide a gentle approach of treating oral health, including gum disease, and are capable of reversing gum disease over time and without the use of non-natural products. The methods are consistent with the practice of biomimetic dentistry and may readily be employed in the practice of biomimetic dentistry.

### DETAILED DESCRIPTION OF THE DISCLOSURE

The best mode for carrying out the present disclosure is presented in terms of its preferred embodiments described herein. The preferred embodiments described herein detail for illustrative purposes are subject to many variations. It is understood that various omissions and substitutions of equivalents are contemplated as circumstances may suggest or render expedient, but are intended to cover the application or implementation without departing from the spirit or scope of the present disclosure.

The terms “a” and “an” herein do not denote a limitation of quantity, but rather denote the presence of at least one of the referenced items.

In an embodiment, a method for administering improved hygiene services and a botanical and biomimetic treatment of dental and periodontal conditions comprises the steps of (1) inquiring of a patient as to whether the patient has any allergy to botanical products, (2) applying an aerosol spray to a quadrant of the patient's oral cavity, said oral spray preferably comprising a botanical composition such as a herbal oil, baking soda, sea salt, or aloe vera gel. The application of the spray to the patient is done prior to using instrumentation on the patient's teeth (to loosen plaque and tartar from the patient's teeth) or gums (to open the gum tissue). Also prior to instrumentation, the instruments (such as traditional dental picks and scrapers) may be dipped in clove oil instead of being treated with alcohol. The use of clove oil reduces the instance of drying out the gum tissue, which issue is observed when instrumentation is treated with alcohol. The clove oil also has a numbing effect on the gum tissue to reduce patient discomfort and has antimicrobial properties. After instrumentation (such as usage of traditional dental picks and scrapers) to remove plaque and tartar from the oral cavity, the gum tissue may be left raw or irritated from the instrumentation. Accordingly the method thereafter utilizes a brush to which at least one herbal oil has been applied prior to using the brush on the patient. The brush is used to massage the patient's teeth and gums, which massaging tightens the gum tissue, which reduces the irritation and rawness and restores the tissue to a stronger state. The comfortability of the brush reduces patient anxiety and discomfort. The method is repeated for the remaining quadrants of the patient's mouth to complete the treatment. In another embodiment, the method includes administering a tooth polish composition to the patient via a traditional dental polish instrument (that applies polish to a patient's teeth by way of a rotating or vibrating cup that is disposed at the end of the polish instrument. In such an embodiment, instead of traditional granular dental polish composition, the polish of the present method is comprised from at least one herbal oil, the texture of which is less disturbing to the patient than traditional dental polish compositions. The oil or oils used in the process may be left in the patient's oral cavity (as opposed to being expectorated by the patient) so that the healing and cleansing effects of the oil or oils may have a duration that extends after the initial application of such oil or oils.

In another embodiment, a method for administering improved hygiene services and a botanical and biomimetic treatment of dental and periodontal conditions may be practiced by a patient at home (as opposed to being administered by a dental professional at a dental office). In such an embodiment, the method comprises the steps of (1) rinsing the oral cavity with one of a solution of sea salt or an oil pulling mouthwash to loosen plaque and tartar and to activate gum tissue, (2) brushing the teeth and gums with a brush to which at least one herbal oil has been applied, (3) flossing the teeth and gums, and or cleaning the teeth with teeth sticks (4) applying an aerosol spray to a quadrant of the patient's oral cavity, said oral spray preferably comprising a botanical composition such as a herbal oil or oils, and (5) massaging the gums with the oil or oils. In an embodiment, if gums are inflamed, the patient may optionally apply the oil to a piece of gauze and then wrap the inflamed area with the oil-infused gauze.

The method includes providing products for the dental professional to administer treatment to the patient, and providing products for the patient to self-administer treatment. Products for administration of the method include tooth wrap kits (which kits may be appropriately-sized pieces of gauze, which pieces may be pre-infused with a herbal oil or oil(s), or which pieces may be accompanied by separately-packaged and appropriately-sized volumes of such oil or oil(s). Another product includes a brush, which brush may be used to massage gums and teeth. Another product includes a toothpaste composition, which composition may, in an embodiment, comprise a herbal oil such as camellia oil, coconut oil or lavender oil, which oil assists in naturally whitening the surface of teeth. It was observed that the camellia oil closes dental tubules and therefore has a de-sensitizing effect. The lavender oil reduces inflammation of the gum tissue. The toothpaste may be in the form of a traditional paste or may be in a foaming form. Another product includes an aerosol dispenser, which dispenser may be filled with a herbal oil or oils for application to the patient's oral cavity. A tongue cleansing product, comprising a herbal oil may also be provided, with a more rigid, but still gentle brush for applying the cleansing product to the tongue. Yet another product is a container for storage of each individual herbal oil utilized in the method.

Another product is a mouthwash, which mouthwash is comprised of a herbal oil and may be ingested temporarily by the patient to perform an “oil-pulling” process of an exemplary embodiment of the method. Trays may also be provided, which trays may be filled with camellia oil, coconut oil or lavender oil to allow the patient to perform as tooth-whitening procedure that is separate from the toothpaste/brushing process. A separate container containing clove oil may be provided, which clove oil may be used to relieve pain in the teeth and/or gums as needed in performing the method.

The method may also include tooth sticks and floss as cleaning products. The floss may be coated and the tooth sticks may be infused with a herbal oil as part of the method. In another embodiment, the floss may be coated and the tooth sticks may be infused with a fruit or mint oil to provide for a pleasing flavor for patients. The gauze of the tooth wrap kits may also be similarly infused and flavored.

In an embodiment, the herbal oils utilized in the method may comprise camellia oil, lavender oil, coconut oil, Echinacea oil, rose hip oil, oregano oil, clove oil, calendula oil, chamomile herb oils, or any other herbal oil with beneficial effects on a patient's oral cavity. For example, the rose hip oil was found to add vitamin C to gum tissue to reduce inflammation, and the calendula oil also had an anti-inflammatory effect. In an embodiment, the mouthwash may be composed of oils such as camellia oil, rose hip oil, lavender oil, coconut oil. peppermint oil, sage oil, anise oil, coenzyme 10 extract, melaleuca altenfolia, propols extract, eucalyptus oil, rosemary oil, tea tree oil, baking soda, sea salt, aloe vera gel and other natural oils and herbs. It will be apparent that the products are packaged with instructions that inform the patient of how to practice the method at home. The anti-oxidants present in the oils assist in reducing inflammation of gum tissue, which, taken with the brush and toothpaste steps of the method, provides natural method of reversing gum disease and prevention of tooth loss.

The herbal oils and compositions used in the method are specifically free of chemical additives, such as peroxides or titanium dioxide, which omission precludes the method from presenting adverse effects to a user's oral cavity through use of the method. By excluding peroxides and similar chemicals therefrom, the compositions of the method do not pose any health risks to a user's gum tissue. Thus, the disclosed method does not require the user to protect his or her gums from being contacted by the composition while the user is applying the compositions of the method to his or her teeth. The oils may be left in the patient's oral cavity after treatment of the teeth and gums (as opposed to being expectorated by the patient) so that the beneficial properties of the oils (such as pain relief or anti-inflammation) can work on the teeth and gums for an extended period. Further, the characteristics of the compositions used in the disclosed treatment methods are similar to the conditions of a patient's oral environment such that the compositions mimic such conditions to provide more efficient and less negatively impactful treatments to a patient and can therefore be used as part of a broader biomimetic treatment regime. In short, the methods mimic natural ways of healing dentition, thereby providing a natural biomimetic dental solution for treating oral conditions and reducing fear or apprehension in the patient.

The compositions may be provided as oral formulations, as powders, gels, oils, liquids, sprays, pastes, tooth powders, tooth gels, fluids, rinses, mouthwash, toothpaste, and the like.

The methods disclosed herein may be practiced by a dental professional on a patient in a dental office, or may be practiced by the patient with the written instructions provided with the products used in the method. In an embodiment, the patient practices the method at least once daily. In addition to reversing gum disease, it will be apparent that daily practice of the method reduces the adherence of plaque and tartar on the patient's teeth.

The present disclosure offers the following advantages. The oils and compositions clean teeth and gums using natural ingredients, thereby reducing tooth discoloration and inflammation of the gums without the need for chemical additives that may otherwise negatively affect a patient's oral tissues or compromise the patent's health or comfort. The natural ingredients are safely ingestible by patient, which decreases the risk that a patient may harm or poison him or herself in practicing the method. The instruments involved in the method are configured not to irritate gums in the process, thus reducing patient discomfort and the risk of infection as compared to prior art dental hygiene programs.

The overall safety and gentleness of the method solves the problem of patient anxiety in visiting a dental professional for treatment and therefore increases the likelihood that the patient will continue to obtain treatment from a dental professional and otherwise not hesitate to maintain a routine checkup and treatment schedule with a dental professional.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the disclosure and various embodiments with various modifications as are suited to the particular use contemplated.

What is claimed is:

1. A method for administering improved hygiene services and a botanical and biomimetic treatment of dental and periodontal conditions comprises
(1) inquiring of a patient as to whether the patient has any allergy to botanical products,
(2) applying an aerosol spray to a first quadrant of the patient's oral cavity,
(3) applying clove oil to dental instruments prior to use of the instruments on a patient,
(4) subjecting the patient to the instruments,
(5) applying herbal oil to a brush, and
(6) using the brush to massage the gums and teeth of the first quadrant of the patient's oral cavity,
(7) repeating steps (1) through (6) for the remaining quadrants.

2. The method of claim 1, wherein, the composition of the aerosol spray comprises one of an herbal oil, baking soda, sea salt, and aloe vera gel.

3. A method for administering improved hygiene services and a botanical and biomimetic treatment of dental and periodontal conditions comprises
(1) rinsing the oral cavity with one of a solution of sea salt or an oil pulling mouthwash,
(2) brushing the teeth and gums with a brush to which at least one herbal oil has been applied,
(3) flossing the teeth and gums,
(4) applying an aerosol spray to a quadrant of the patient's oral cavity, said oral spray preferably comprising a botanical composition such as a herbal oil or oils, and
(5) massaging the gums with the oil or oils.

4. The method of claim 3, wherein the method further comprises applying an oil to a piece of gauze and wrapping a selected area of gums with the oil-infused gauze.

5. The method of claim 3, wherein the oil of step (2) comprises one of camellia oil, coconut oil, and or lavender oil.

6. The method of claim 3, wherein the method further comprises administration of an aerosol spray comprising herbal oil or oils for into the patient's oral cavity.

7. The method of claim 3, wherein the method further comprises applying a herbal oil cleansing product to the tongue of a patient.

8. The method of claim 3, wherein the method further comprises applying a mouthwash to the patient, wherein the mouthwash comprises at least one of camellia oil, rose hip oil, lavender oil, coconut oil. peppermint oil, sage oil, anise oil, coenzyme 10 extract, melaleuca altenfolia, propols extract, eucalyptus oil, rosemary oil, tea tree oil, baking soda, sea salt, and aloe vera gel.

9. The method of claim 3, wherein the method further comprises cleaning the oral cavity with floss that has been infused with an herbal oil.

10. A method for administering improved hygiene services and a botanical and biomimetic treatment of dental and periodontal comprises administering a tooth polish composition to the patient via a dental instrument, wherein the polish comprises at least one herbal oil.
